(19) United States
(12) Patent Application Publication
(10) Pub. No.: US 2011/0046141 A1
(43) Pub. Date: Feb. 24, 2011

(54) USE OF PDE5 INHIBITORS FOR TREATING CIRCADIAN RHYTHM DISORDERS

(75) Inventors: Diego Andrés Golombek, Buenos Aires (AR); Patricia Verónica Agostino, Quilmes - Prov. De Buenos Aires (AR); Santiago Andrés Plano, Bernal - Prov. De Buenos Aires (AR); Gabriela Alejandra Ferreyra, Bethesda, MD (US)

Correspondence Address: YOUNG & THOMPSON, 209 Madison Street, Suite 500, Alexandria, VA, 22314, US

(73) Assignees: CONSEJO NACIONAL DE INVESTIGACIONES CIENTIFICAS Y TECNICAS (CONICET), Buenos Airaes (AR); UNIVERSIDAD NACIONAL DE QUILMES, Prov. De Buenos Aires (AR)

(21) Appl. No.: 12/128,795

(22) Filed: May 29, 2008

Related U.S. Application Data
(60) Provisional application No. 60/967,567, filed on Sep. 5, 2007.

(51) Int. Cl. A61K 31/519; A61K 31/53; A61K 31/4985; A61P 25/00

(52) U.S. Cl. 514/243; 514/252.16; 514/261.1; 514/250

(57) ABSTRACT

A method of altering circadian rhythm in a mammal is provided. In certain embodiments, the method comprising: administering to the mammal a PDE5 inhibitor, e.g., sildenafil, vardenafil, tadalafil or zaprinast. The method may be employed to prevent a circadian rhythm disorders including, but not limited to transmeridian flight disorder (i.e., “jet-lag”), shiftwork-related disorder, seasonal affected disorder and insomnia by phase delay or phase advance.

### BACKGROUND

The mammalian circadian clock in the brain conveys 24-hr rhythmicity to sleep-wake cycles, temperature, locomotor activity and virtually all other behavioral and physiological processes. In order for these cycles to be adaptive, they must be synchronized, or entrained, to the 24-hr light/dark cycle produced by the rotation of the Earth. Air travelers who cross several time zones are commonly affected by jet-lag symptoms which include impaired sleep, mood and cognitive performance which result from the body's internal rhythms being out of step with the day-night cycle at the destination. Circadian rhythm sleep disorders are a group of pathologies characterized by an internal de-synchronization between a person's biological clock and their environmental 24-hr schedule. Winter depression and delayed sleep phase syndrome (DSPS) belong to this class of disorders.

### SUMMARY OF THE INVENTION

A method of altering circadian rhythm in a mammal is provided. In certain embodiments, the method comprising: administering to the mammal a PDE5 inhibitor, e.g., sildenafil, vardenafil, tadalafil or zaprinast. The method may be employed to prevent a circadian rhythm disorder including, but not limited to transmeridian flight disorder (i.e., “jet-lag”), shiftwork-related disorder, seasonal affected disorder and insomnia by phase delay or phase advance.

Suitable PDE5 inhibitors for use in the subject methods include, but are not limited to a pyrazolo (4,3-d)pyrimidin-7-one; isomeric pyrazolo (3,4-d)pyrimidin-4-one; a quinazolin-4-one; a pyrido (3,2-d)pyrimidin-4-one; a purin-6-one; and pyrazolo (4,3-d)pyrimidin-4-one, e.g., 3-ethyl-5-(5-(4-ethylpiperazin-1-ylsulphonyl)-2-n-propoxyphenyl)-2-(pyrid-in-2-yl)methyl-2,6-dihydro-7H-pyrazolo(4,3-d)pyrimidin-7-one (sildenafil), (2-[2-ethoxy-5-(4-ethylpiperazine-1-sulfonyl)-phenyl]-5-methyl-7-propyl-3-H-imidazo[5,1-f][1,2,4]triazin-4-one) (vardenafil), or Pyrazino[1′,2′:1,6]pyrido[3,4-b]indole-1,4-dione,6-(1,3-benzodioxol-5-yl)-2,3,6,7,12,12a-hexahydro-2-methyl-,(6R, 12aR)-6R-trans)-6-(1,3-benzodioxol-5-yl)-2,3,6,7,12,12a-hexahydro-2-methyl-1-pyrazino[1′,2′:1,6]pyrido[3,4-b]indole-1,4-dione (tadalafil).

### BRIEF DESCRIPTION OF THE FIGURES

FIG. 1 is four panels of graphs showing the effects of sildenafil on circadian reentrainment. Double-plotted actograms of hamster wheel-running activity showing reentrainment to a 6-hr advance of the LD cycle after the injection of (A) vehicle and (B) sildenafil (3.5 mg/kg, i.p.) at ZT18 on the day of the cycle change. Periods of darkness are shaded in gray. (C) Summary of phase advances (min) on each day after the change in the to LD cycle (n=6 animals/group, means±S.E.M.). Open and filled circles indicate saline and sildenafil, respectively, ***p<0.001, **p<0.01, *p<0.05, Student's t-test. (D) Dose-response curve for sildenafil effects (mean±S.E.M, n=6). ***p<0.001, *p<0.05, ANOVA followed by Tukey's test.

FIG. 2 is two panels of graphs showing the effects of sildenafil on light-induced phase advances. (A) Double-plotted actograms of hamster wheel-running activity showing vehicle or sildenafil (3.5 mg/kg, i.p.) injection 45 min. before a light pulse at CT 18. Light stimulation is indicated by a star. Activity onsets are indicated by straight lines drawn over the actograms. (B) Quantification of phase advances (mean±S.E.M., n=5), *p<0.05, Student's t-test.

FIG. 3 shows the effects of sildenafil on phase delays. (A) Effect on reentrainment of wheel-running activity rhythm following a 6-hr phase delay of the LD cycle. Double-plotted actograms of hamster wheel-running activity showing reentrainment to a 6-hr delay of the LD cycle after the injection of vehicle (top) and sildenafil (3.5 mg/kg, i.p., middle). Injections of either saline or sildenafil were given at ZT 14 on the day of the cycle change (white star). Bottom: mean±S.E.M. (n=4 animals per group) of days needed for reentrainment. (B) Effect of sildenafil on light-induced phase delays following light pulses at CT 14. Representative actograms showing vehicle (top) or sildenafil (middle) injections. Bottom graph: Mean±S.E.M. (n=4 animals per group).

FIG. 4 shows eight panels of confocal images showing neuronal localization of cGMP in the SCN. Combination of single confocal images for cGMP and GFAP or NeuN staining. (A) cGMP; (B) glial fibrillary acidic protein, GFAP; (C), double-labeling of cGMP-GFAP; (D) higher magnification of (C) shows no colocalization between cGMP and GFAP; (E) cGMP; (F) neuron-specific nuclear protein, NeuN; (G) double-labeling of cGMP-NeuN; (H) higher magnification of (G) shows cells with both cGMP (cytoplasmic) and NeuN (nuclear), suggesting neuronal expression of cGMP in the SCN. Scale bars: 100 μM (A-C, E-G) and 20 μM (D, H).

FIG. 5 is a graph showing the effects of sildenafil, vardenafil and tadalafil on phase advance after a light pulse at CT18 (15 min, 50 lux).

### DEFINITIONS

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with general dictionaries of many of the terms used in this invention. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

The term “PDE5 inhibitor” refers to a class of compounds that inhibits the human cyclic guanosine 3′,5′-monophosphate phosphodiesterase 5 (PDE5). Such drugs, exemplified by sildenafil, vardenafil, tadalafil and zaprinast, are well known for their effects on sexual (e.g., erectile) disfunction.

The term “circadian rhythm disorder” refers to disorders that result in an internal de-synchronization between a person's biological clock and their environmental 24-hour schedule subjects circadian rhythm. Such disorders include, but are not limited to, a transmeridian flight disorder (i.e., “jet-lag”), shiftwork-related disorder, seasonal affected disorder, and insomnia caused by phase delay or phase advance.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease.

“Treatment”, as used herein, covers any treatment of a disorder in a mammal, particularly in a human, and includes: (a) preventing the disorder from occurring in a subject which may be i. predisposed to the disorder but has not yet been diagnosed as having it or ii. expected to contract the disorder; (b) inhibiting the disorder, i.e., arresting its development; and (c) relieving the disorder, i.e., causing regression of the disease and/or relieving one or more disease symptoms. “Treatment” is also meant to encompass delivery of an agent in order to provide for a pharmacologic effect, even in the absence of a disease or condition.

“Subject”, “host” and “patient” are used interchangeably herein, to refer to an animal, human or non-human, susceptible to or having a circadian rhythm disorder. Generally, the subject is a mammalian subject. Exemplary subjects include, but are not necessarily limited to, humans, cattle, sheep, goats, pigs, dogs, cats, and horses, with humans being of particular interest.

### DETAILED DESCRIPTION

As noted above, a method of altering circadian rhythm in a mammal is provided. In certain embodiments, the method comprising: administering to the mammal a PDE5 inhibitor, e.g., sildenafil, vardenafil, tadalafil or zaprinast. The method may be employed for the treatment of (including the prevention of) a circadian rhythm disorder.

PDE5 inhibitors alter the phase of (i.e., entrain) the circadian rhythm of a subject, independent of the sleep-wake cycle of the subject. As such, a PDE5 inhibitor may be administered to a subject having a circadian rhythm disorder, or in anticipation of the development of such a disorder. Exemplary disorders include, but are not limited to, jet-lag, winter depression (or so-called “seasonal affective disorder”), delayed sleep phase syndrome (DSPS), and shift-related disorders. As such, suitable subjects include, but are not limited to intercontinental travelers, aircraft pilots, shift workers (e.g., subjects that work the night shift and sleep during the day or have irregular shifts), and people that are affected by winter depression (e.g., subjects that live in or close to the arctic circles or in regions of limited winter light), for example. In certain embodiments, the PDE5 inhibitor may be administered late at night, e.g., immediately prior to the subject going to sleep.

In certain cases, a single dose of the PDE5 inhibitor is administered. In other cases, a sufficient number of doses is administered until the subject's circadian rhythm is re-set. In particular embodiments, the PDE5 inhibitor may be administered in conjunction with another drug, e.g., melatonin, that modulates circadian rhythm. In a particular embodiment, the PDE5 inhibitor may be administered as a single dose, prior to a phase change (e.g., before travel). In other embodiments, the PDE5 inhibitor may be administered in conjunction with light (photic) stimulation.

### PDE5 Inhibitors

Many PDE5 inhibitors are known in the art and are suitable for use in the subject methods. In certain embodiments, the PDE5 inhibitor used in the instant methods may be a cGMP specific PDE5 inhibitor (cGMP PDE5 inhibitor). Such inhibitors include: the pyrazolo[4,3-d]pyrimidin-7-ones disclosed in EP-A-0463756; the pyrazolo[4,3-d]pyrimidin-7-ones disclosed in EP-A-0526004; the pyrazolo[4,3-]pyrimidin-7-ones disclosed in published international patent application WO 93/06104; the isomeric pyrazolo[3,4-d]pyrimidin-4-ones disclosed in published international patent application WO 93/07149; the quinazolin-4-ones disclosed in published international patent application WO 93/12095; the pyrido[3,2-d]pyrimidin-4-ones disclosed in published international patent application WO 94/05661; the purin-6-ones disclosed in published international patent application WO 94/00453; the pyrazolo[4,3-d]pyrimidin-7-ones disclosed in published international patent application WO 98/49166; the pyrazolo[4,3-d]pyrimidin-7-ones disclosed in published international patent application WO 99/54333; the pyrazolo[4,3-d]pyrimidin-4-ones disclosed in EP-A-0995751; the pyrazolo[4,3-d]pyrimidin-7-ones disclosed in published international patent application WO 00/24745; the pyrazolo[4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124; the pyrazolo[4,3-d]pyrimidin-7-ones disclosed in published international application WO 01/27112; the pyrazolo[4,3-d]pyrimidin-7-ones disclosed in published international application WO 01/27113; the compounds disclosed in EP-A-1092718; and the compounds disclosed in EP-A-1092719.

Other PDE5 inhibitors include: 5-[2-ethoxy-5-(4-methyl-1-piperazinylsulphonyl) phenyl]-1-methyl-3-n-propyl-1,6-dihydro-7H-pyrazolo[4,3-d]pyrimidin-7-one (sildenafil), also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyrid-in-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); (+)-3-ethyl-5-[5-(4-ethylpiperazin-1-ylsulphonyl)-2-(2-methoxy-1(R)-methy-lethoxy)pyridin-3-yl]-2-methyl-2,6-dihydro-7H-pyrazolo[4,3-d]pyrimidin-7-o-ne, also known as 3-ethyl-5-{5-[4-ethylpiperazin-1-ylsulphonyl]-2-([(1R)-2-methoxy-1-methyl-ethyl]oxy)pyridin-3-yl}-2-methyl-2,6-dihydro-7H-pyrazolo[4,3-d]pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4-,3-d]pyrimidin-5-yl]-3-pyridylsulphonyl}-4-ethylpiperazine (see WO 01/27113, Example 8); 5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15); 5-[2-Ethoxy-5-(4-ethylpiperazin-1-ylsulphonyl)pyridin-3-yl]-3-ethyl-2-phe-nyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 0/27112, Example 124); 5-(5-Acetyl-2′ butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8; 2-[2-ethoxy-5-(4-ethyl-piperazin-1-yl-1′-sulphonyl)-phenyl]-5-methyl-7-pr-opyl-3H-imidazo[5,1-f][1,2,4]-triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and the compound of example 11 of published international application WO93/07124; and compounds 3 and 14 from Rotella D P, J. Med. Chem., 2000, 43, 1257.

Still other type PDE5 inhibitors useful in conjunction with the present method include: 4-bromo-5-(pyridylmethylamino)-6-[3-(4-chlorophenyl)-propoxy]-3-(2H)pyridazinone; 1-[4-[(1,3-benzodioxol-5-ylmethy)amino]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-met-hyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,-1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl)propoxy)-3-(2H) pyridazinone; 1-methyl-5(5-morpholinoacetyl-2-n-propoxyphenyl)-3-n-propyl-1,6-dihydro-7-H-pyrazolo(4,3-d)pyrimidin-7-one; 1-[4-[(1,3-benzodioxol-5-ylmethyl) amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

In certain case, the PDE5 inhibitor is 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine) (sildenafil, sold under the tradename of VIAGRA™, or pharmaceutically acceptable salt thereof, especially sidenafil citrate). A process for its preparation is described in U.S. Pat. No. 6,207,829.

In another case, the PDE5 inhibitor 2-[2-ethoxy-5-(4-ethyl-piperazine-1-sulfonyl)-phenyl]-5-methyl-7-propy-1-3H-imidazo[5,1-f][1,2,4]-triazin-4-one (vardenafil, sold under the tradename of LEVITRA™) (see e.g. U.S. Pat. No. 6,362,178).

In another case, the PDE5 inhibitor is Tadalafil (Pyrazino[1′,2′:1,6]pyrido[3,4-b]indole-1,4-dione,6-(1,3-benzodioxol-5-yl)-2,3,6,7-,12,12a-hexahydro-2-methyl-,(6R, 12aR)-6R-trans)-6-(1,3-benzodioxol-5-yl)-2,3,6,7,12,12a-hexahydro-2-methyl-1-pyrazino[1′,2′:1,6]pyrido[3,4-b]indole-1,4-dione, sold under the tradename CIALIS™) to (U.S. Pat. Nos. 5,859,006, 6,140,329).

Other PDE5 inhibitors suitable for the present method include zaprinast, FR226807, T-1032, KF31327, UK369003, TA1790, DA8159 (Rotella D P. Phosphodiesterase 5 inhibitors: current status and potential applications. Nature Reviews. Drug Discovery. 1(9):674-82, 2002.), and UK122764 (Turko et al., 1999, Inhibition of cyclic CGP-binding cyclic GMP specific phosphodiesterase (type 5) by sildenafil and related compounds. Molecular Pharmacology 56: 124-130).

Other PDE5 inhibitors are discussed in Rotella et al., N-3-substituted imidazoquinazolinones: potent and selective PDE5 inhibitors as potential agents for treatment of erectile dysfunction. Journal of Medicinal Chemistry. 43(7):1257-63, 2000. Rotella et al., Optimization of substituted N-3-benzylimidazoquinazolinone sulfonamides as potent and selective PDE5 inhibitors. Journal of Medicinal Chemistry. 43(26):5037-43, 2000. Kim et al., Synthesis and Phosphodiesterase 5 Inhibitory Activity of New 5-Phenyl-1,6-dihydro-7H-pyrazolo[4,3-d]pyrimidin-7-one derivatives Containing an N-Acylamido Group on a Phenyl Ring. Bioorganic & Medicinal Chemistry 9 1895-1899, 2001.

The suitability of any particular PDE5 inhibitor can be readily determined by evaluation of its potency and selectivity using methods described in the scientific literature or known to those skilled in the art followed by evaluation of its toxicity, absorption, metabolism, pharmacokinetics, etc. in accordance with standard pharmaceutical practice. The PDE5 inhibitor may be specific for PDE5, or may be non-specific for PDE5 in that it affects other GMP phosphodiesterases.

### Routes of Administration

Specific methods by which the PDE5 inhibitors, their pharmaceutically acceptable salts and pharmaceutically acceptable solvates, when used in accordance with the invention, may be administered for human clinical or veterinary use, including oral administration by capsule, bolus, tablet or drench, topical administration as an ointment, pour-on, dip, spray, mousse, shampoo, collar or powder formulation, or, alternatively, they can be administered by injection (e.g. subcutaneously, intramuscularly or intravenously), or as an implant. Such formulations may be prepared in a conventional manner in accordance with standard practices well-known to those skilled in the art.

Alternatively, in veterinary use, the PDE5 inhibitors, their pharmaceutically acceptable salts, and pharmaceutically acceptable solvates, when used in accordance with the invention, may be administered with an animal feedstuff and for this purpose a concentrated feed additive or premix may be prepared for mixing with the normal animal feed.

The PDE5 inhibitors, their pharmaceutically acceptable salts, and pharmaceutically acceptable solvates, when used in accordance with the invention, can be administered orally, buccally or sublingually in the form of tablets, capsules (including soft gel capsules), ovules, elixirs, solutions or suspensions, which may contain flavoring or coloring agents, for immediate-, delayed-, modified-, or controlled-release such as sustained-, dual-, or pulsatile delivery applications. The PDE5 inhibitors, their pharmaceutically acceptable salts, and pharmaceutically acceptable solvates, when used in accordance with the invention, may also be administered via fast dispersing or fast dissolving dosage forms or in the form of a dispersion. Suitable pharmaceutical formulations of the PDE5 inhibitors, their pharmaceutically acceptable salts, and pharmaceutically acceptable solvates, when used in accordance with the invention, may be in coated or uncoated form as desired.

Such tablets may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and talc may be included. Physiologically acceptable carriers, excipients, or stabilizers are known to those skilled in the art (see Remington's Pharmaceutical Sciences, 17th edition, (Ed.) A. Osol, Mack Publishing Company, Easton, Pa., 1985). Acceptable carriers, excipients or stabilizers are nontoxic to recipients at the dosages and concentrations employed, and include buffers such as phosphate, citrate, and other organic acids; hydrophobic oils derived from natural or synthetic sources; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

Excipients in this regard include lactose, starch, cellulose, milk sugar or high molecular weight polyethylene glycols. For aqueous suspensions and/or elixirs, the compounds of the invention may be combined with various sweetening or flavoring agents, coloring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients maybe present both within the dosage form i.e. within the matrix, and/or on the dosage form i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The PDE5 inhibitors, their pharmaceutically acceptable salts, and pharmaceutically acceptable solvates, when used in accordance with the invention, can also be administered parenterally, for example, intravenously, intra-arterially; intraperitoneally, intrathecally, intraventricularly, intraurethrally, intravaginally, intrasternally, intracranially, intramuscularly or subcutaneously, or they may be administered by infusion or needleless injection techniques. For such parenteral administration they are best used in the form of a sterile aqueous solution that may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

The dosage ranges for the administration of pharmaceutical composition of the invention are those large enough to produce the desired effect. The dosage should not be so large as to cause adverse side effects, such as unwanted cross-reactions, anaphylactic reactions, and the like. Generally, the dosage will vary with the age, condition, sex and extent of condition of the patient and can be determined by one of skill in the art. The dosage can be adjusted by the individual physician in the event of any complication.

In certain embodiments, the PDE5 inhibitor will be administered (e.g., a tablet) at a dose of 10 mg to 1 g, e.g., 25 mg to 250 mg.

In order to further illustrate the present invention and advantages thereof, the following specific examples are given with the understanding that they are being offered to illustrate the present invention and should not be construed in any way as limiting its scope.

### EXAMPLES

Abbreviations: CT, circadian time; LD, light-dark cycle; PDE, phosphodiesterase; SCN, suprachiasmatic nuclei; ZT, zeitgeber time

### Materials and Methods

Animals. Male adult (3-4 month-old) Syrian hamsters (Mesocricetus auratus) were raised and maintained in a 14:10 h light-dark cycle (L:D, lights on at 0600 h), with food and water ad libitum and room temperature set at 20±2° C. All animal procedures were performed in strict accordance with NIH rules for animal care and maintenance.

Activity rhythm recording. For the resynchronization experiment, animals were transferred to individual cages equipped with a running wheel (17 cm. diameter) and with light intensity averaging 200 lux at cage level. Running-wheel activity was continuously recorded for each animal using a digital system that registers wheel revolutions and stored at 5-min intervals for further analysis. Animals were initially maintained under a 14:10-h is light-dark cycle (LD, lights on at 1900 h) for at least 10 days. Then, hamsters were subjected to an abrupt 6-h advance in the phase of the LD cycle. On the day of the phase shift, intraperitoneal (i.p) injection of sildenafil or vehicle was given at zeitgeber time (ZT) 18, defining ZT 12 as the time of lights off. Time for reentrainment to the new LD cycle was defined as the time it took for each animal to adjust its activity onset with the new cycle (onset at the new time of lights off±15 minutes). Daily onsets of activity were determined. Briefly, activity onset was defined as the 10 min-bin that contained at least 80 wheel revolutions, followed by another bin of at least another 80 wheel revolutions within 40 min. The effect of 1, 3.5 or 10 mg/kg sildenafil—extracted from commercial preparations according to Francis et al. (Int. J. Impotence Res. 2003 15: 369-372) was tested at ZT 14 or 18 in comparison to vehicle administration (sterile saline).

For light pulse experiments, hamsters were placed in constant dark (DD) conditions, and exposed to a 15-min white light pulse of 50 lux at either circadian time (CT) 14 or 18 (with CT 12 defined as the onset of wheel running activity in DD). Phase shifts were calculated by fitting a line (by three observers masked to the experimental procedure) through activity onsets 5 days prior and between 5 and 15 days after light exposure and then comparing these two lines on the day of the light pulse. Hamsters received an i.p. injection of either drug or vehicle 45 min before light stimulation.

RT-PCR. Total RNA from hamster SCN, kidney, spleen and heart was isolated in accordance to standard procedures (Trizol, Life Technologies). cDNA was synthesized from 3 μg of RNA using the SuperScript™ First-Strand Synthesis System for RT-PCR (Invitrogen). Specific oligonucleotide primers were as follows: forward primer 5′-CAGGAAATGGTGGGACCTTC (SEQ ID NO:1) and reverse primer 5′-AAGGCTTCCAGGAACTGCTC (SEQ ID NO:2) for PDE5, and forward primer 5′-AGAACTTATCCAGGGCGTGC (SEQ ID NO:3) and reverse primer 5′-TGTCACACAGAAGCAGTGCC (SEQ ID NO:4) for PDE9. GAPDH was used as an internal control; forward primer 5′-CTGCACCACCACCTGCTTAG (SEQ ID NO:5) and reverse primer 5′CTTTCTCCAGGCGACATGTG (SEQ ID NO:6). PCR was performed under the following conditions: denaturing at 94° C. for 10 s, annealing at 62° C. (PDE5) or 60° C. (PDE9) for 15 s and primer extension at 72° C. for 60 s in 35 cycles. PCR products were separated by electrophoresis on a 1% agarose gel and stained with SYBR Green (Molecular Probes). A 619 base pair (bp) fragment for PDE5, a 511 bp fragment for PDE9 and a 309 bp fragment for GAPDH were amplified.

Determination of cGMP levels. Cyclic GMP content was determined using a direct enzyme immunoassay kit (Correlate-EIA #900-014, Assay Designs, Ann Arbor, Mich.). Hamsters received an injection of sildenafil (3.5 mg/kg, i.p.) or vehicle (sterile saline solution) at CT 18 and were sacrificed by decapitation 45-90 min later. The SCN were quickly punched out and immediately frozen in liquid nitrogen in order to avoid endogenous cGMP degradation. Tissue was grounded to a fine powder under liquid nitrogen and then homogenized in 0.1 M HCI. The overnight acetylated format of the kit was used. Endogenous levels of cGMP fitted on the kit sensitivity and were comparable to those previously published by radioimmunoassay methods. In order to precisely compare the effect of sildenafil on cGMP levels, the use of other phosphodiesterase inhibitors, e.g., IBMX, was avoided.

Immunocytochemistry. Animals were deeply anesthetized (ketamine:xylazine, 150:10 mg/kg, i.p.) and perfused intracardially with 0.1 M phosphate-buffered saline (PBS) followed by fixative solution (4% paraformaldehyde in 0.1 M phosphate buffer). After perfusion, brains were dissected and post-fixed overnight at 4° C. in the same solution. Brains were then transferred into 30% sucrose-paraformaldehyde solution for 48 hours. 40 μm coronal sections were cut with a freezing microtome and collected in 0.1 M phosphate buffer. Sections were washed with 0.4% Triton X-100 in 0.01 M PBS (PBS-T). Non-specific binding sites were blocked with 0.1% BSA and 2% normal horse serum (NHS) in PBS-T for 1 h at room temperature. Sections were incubated with cGMP primary antibody 1:4000 (sheep anti formaldehyde-fixed cGMP, diluted in 0.4% PBST with 2% NHS for 48 h at 4° C. Biotinylated anti-sheep IgG (1:2000, Vector Labs) was used as a secondary antibody for 2 h at room temperature, followed by Rhodamine-Avidin incubation (1:500, Vector Labs) for 2 h at room temperature.

For colocalization studies, sections were further incubated overnight at 4° C. with glial fibrillary acidic protein (GFAP, rabbit anti-GFAP 1:1000, Dako) or with neuron-specific nuclear protein (NeuN, mouse anti-NeuN 1:50, Chemicon) antibodies (in 0.4% PBST with 2% NHS). Labelling was visualized using fluorescein anti-rabbit or anti-mouse antibodies (1:500, Vector Labs).

Confocal laser scanning microscopy (Olympus FV-300 microscope) was performed at 488 nm and 543 nm, to reveal fluorescein and rhodamine, respectively. The two channels were scanned separately and merged using Olympus software. Each optical section (0.4 μm) was averaged four times.

### Results

RT-PCR analysis was used to confirm the presence of PDE5 in the hamster SCN. Strong expression of the PDE5 isoform was evident. PDE9 was also present in the SCN, with lower expression than PDE5.

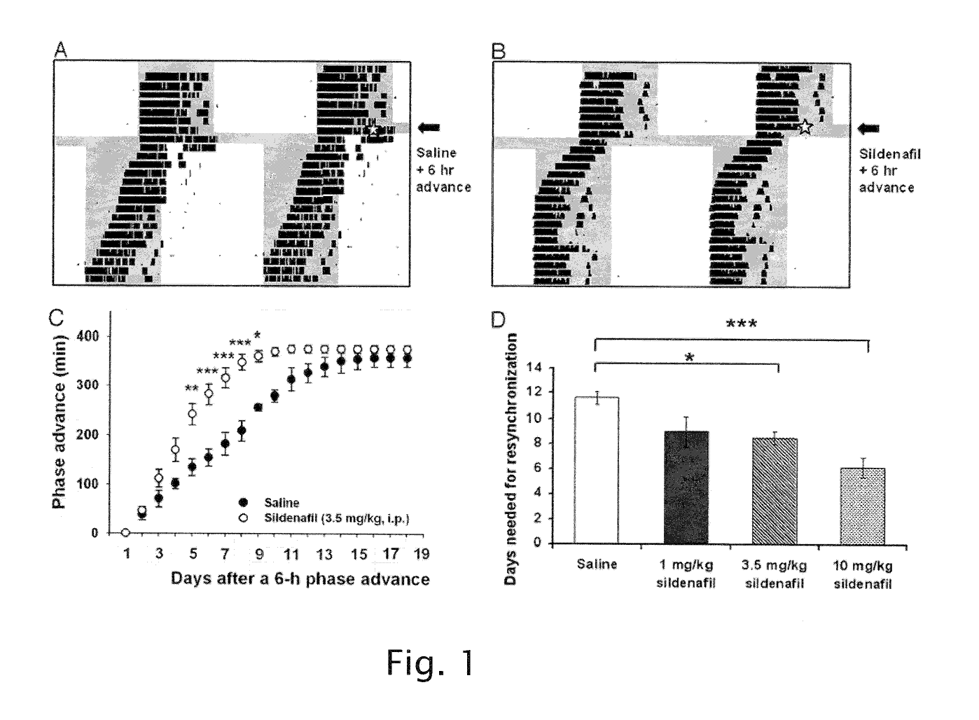

To study the effect of sildenafil on locomotor activity rhythms, hamsters were injected with this compound before an abrupt advance of 6 hours of the light-dark (LD) cycle. When the LD cycle was shifted, the circadian rhythm of running-wheel activity was gradually resynchronized to the new LD cycle. An i.p. injection of 3.5 mg/kg at zeitgeber time 18 (ZT 18, with ZT 12 defined as the time of lights off) of sildenafil on the day of the environmental change significantly accelerated entrainment to the new cycle (FIGS. 1A and 1B). Thus, sildenafil-treated groups took significantly less time to resynchronize to the new LD cycle as compared to the vehicle-treated group (FIG. 1C-D; 12±2 days for saline and 8±1 days for 3.5 mg/kg sildenafil, mean±SD for six animals per group, p<0.05, ANOVA followed by Tukey's test). A lower dose, 1 mg/kg sildenafil, failed to accelerate resynchronization (9±3 days; p>0.05 vs. control), while a dose of 10 mg/kg sildenafil was even more effective on reentrainment rate (6±2 days, p<0.001 vs. control). As shown in FIG. 1D, 10 mg/kg sildenafil decreased reentrainment time by 50%, while 3.5 mg/kg and 1 mg/kg decreased this time by 33 and 25%, respectively. However, the intermediate dose was used for the rest of the experiments because at that dose animals did not manifest the effects of sildenafil-induced penile erections. Indeed, a dose-response study in rats showed that 5 mg/kg/day i.p. was the optimal erectogenic dose of sildenafil.

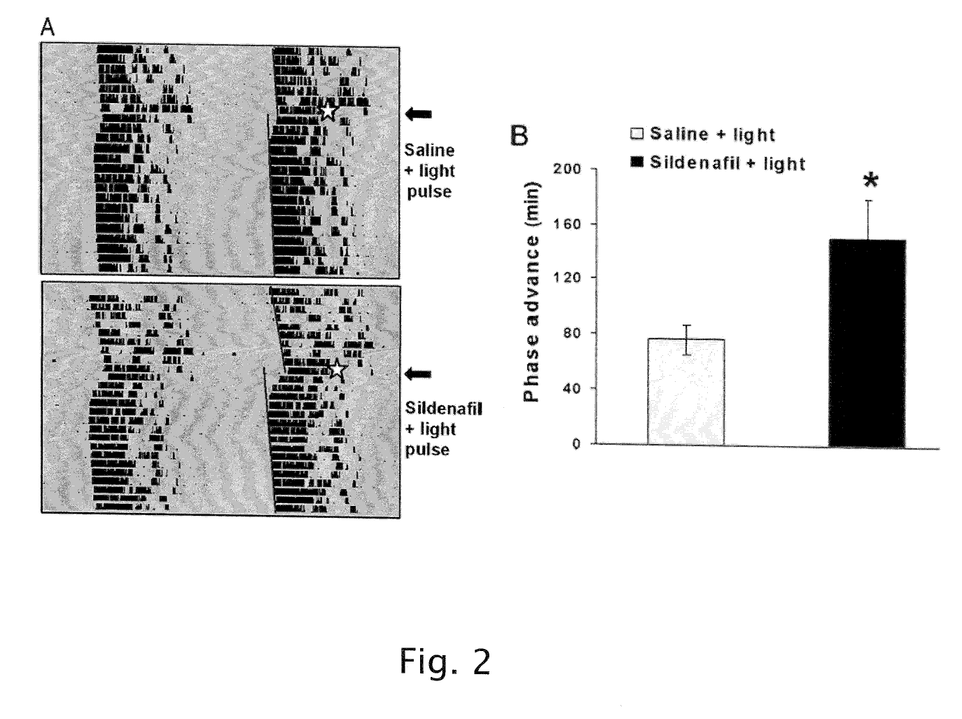

Reentrainment can be considered to be the effect of transient, pulsatile effects of light (usually called non-parametric) as well as tonic, parametric effects of the light cycle. The effect of the PDE5 inhibitor was tested on the well-known non-parametric effects of light, which are defined by phase shifts induced by short light pulses at different times of the day. Sildenafil elicited an increase in light-induced phase advances of activity rhythms when injected 45 min (but not 15 or 90 min) prior to a light pulse at circadian time 18 (CT 18, with CT 12 defined as the time of locomotor activity onset). A 15-min light pulse (50 lux) at CT 18 following vehicle injection induced an average phase advance of 76±23 min, which was increased significantly by a sildenafil injection 45 min prior to the light stimulation (150.4±64.8 min, p<0.05, ANOVA followed by Dunnett's test, FIG. 2), whereas an injection 90 min before the light pulse elicited a phase advance of 123±27 min, which was not significantly different from controls (mean±SD from 5-6 animals per group). Sildenafil alone did not modify activity rhythms. Another cGMP PDE inhibitor, zaprinast (3.5 mg/kg, i.p.) had a similar effect as sildenafil on light-induced phase advances when tested at CT 18 (data not shown).

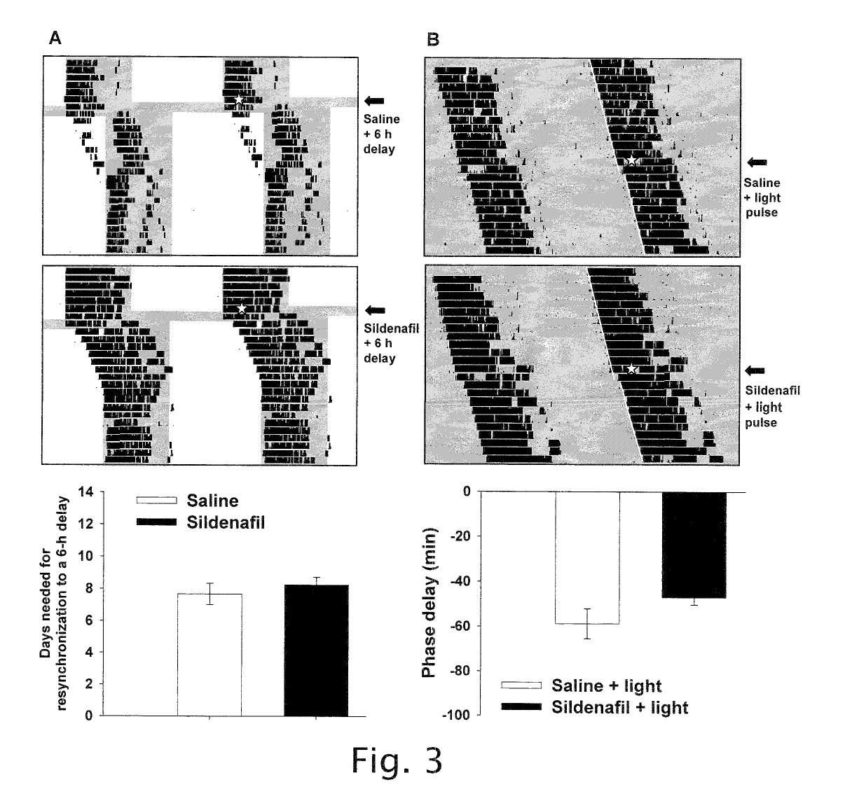

In addition, sildenafil did not affect either reentrainment rates after a delay in the LD cycle (FIG. 3A) nor light-induced phase delays of the circadian locomotor activity rhythm after a light pulse at CT 14 (FIG. 3B).

In order to confirm inhibition of phosphodiesterase by sildenafil, cGMP levels were measured in the hamster SCN using a direct cGMP enzyme immunoassay kit (Assay Designs, Ann Arbor, Mich.). The amount of cGMP at CT 18 was 0.29±0.15 pmol cGMP/mg protein (control values). Administration of 3.5 mg/kg sildenafil induced a two-fold increase in SCN cGMP levels 45 minutes after injection, reaching values comparable to cerebellar cGMP levels, while saline injections had no effect (sildenafil: 0.63±0.08 pmol cGMP/mg protein; saline: 0.37±0.20 pmol cGMP/mg protein; cerebellum 0.70±0.15; values are given as mean±SD, p<0.05, sildenafil vs. control and sildenafil vs. saline, ANOVA followed by Student-Newman-Keuls Multiple Comparisons Test, n=4). Sildenafil injections 90 min before light pulses did not increase cGMP levels in the SCN with respect to controls or saline-treated animals (sildenafil: 0.37±0.20 pmol cGMP/mg protein; saline: 0.26±0.16 pmol cGMP/mg protein, n=4).

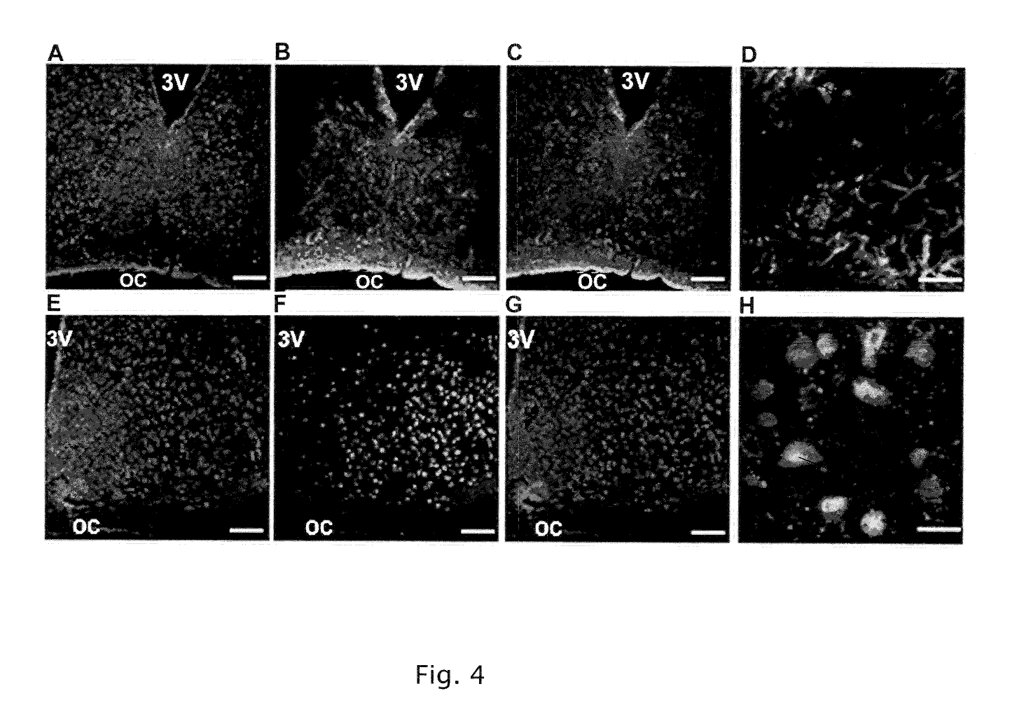

cGMP localization in the SCN was also determined. Distribution of this nucleotide was studied by immunohistochemistry on 40 μm coronal brain sections. cGMP was present in the whole SCN, although more cGMP-like immunoreactivity labeling was observed in the ventral portion of the suprachiasmatic nuclei. Labeling of cGMP was prominent in the cytoplasm. Confocal microscopy showed co-localization of cGMP with the neuron-specific nuclear protein (NeuN) and lack of co-localization with the glial fibrillary acidic protein (GFAP), indicating neuronal localization of cGMP in the SCN (FIG. 4).

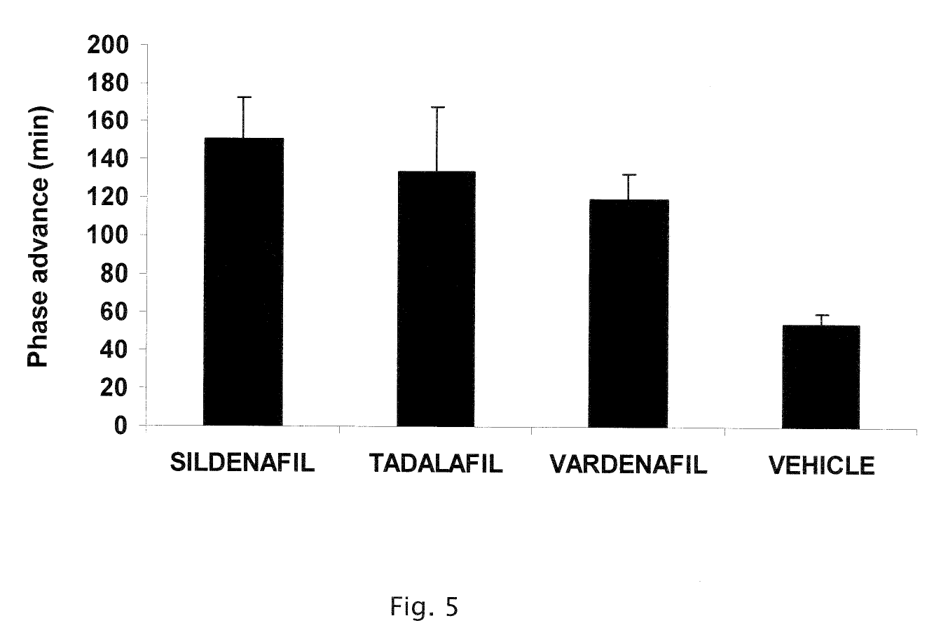

Similar experiments performed using PDE5 inhibitors vardenafil and tadalafil on phase advance after a light pulse yielded similar results as sildenafil (FIG. 5).

In this study, the effects of a selective PDE5 inhibitors on the ability of hamsters to adapt to a 6-h phase change in the LD cycle were determined. The results demonstrate that administration these inhibitors significantly accelerates reentrainment to advancing cycles. Sildenafil increases the response for single light pulses at CT 18, when light induces phase advances. These effects of sildenafil are mediated by a 2-fold increase in SCN cGMP levels 45 min after injection, higher than that previously published with light alone (Ferreyra et al (2001) Am. J. Physiol. 280: R1348-R1355). There is a clear phase specificity for this pathway, since sildenafil had no effect on reentrainment after a phase delay of the light-dark cycle nor on light-induced phase delays under constant dark conditions. Since cGMP levels vary in the SCN under both light-dark and constant dark conditions, the phase dependency of sildenafil administration could be related to this endogenous variation, because cGMP increases would represent differential values with respect to basal levels for the cyclic nucleotide. Our results demonstrate a differential pathway responsible for circadian delay or advance mechanisms (since sildenafil lacked any effects in the early night), as well as a corroboration of a cGMP role in photic entrainment and a specific role for PDE5 in this process. PDE5 inhibitors may be employed for the treatment of certain circadian disorders, such as phase delay or advance of human sleep-wake cycle, jet-lag and shiftwork-related disturbances, and the like.

Although sildenafil induced an increase in light-induced phase advances, the post-treatment circadian period did not change with respect to previous conditions. However, since the slope of the reentrainment curve after sildenafil administration is significantly increased with respect to controls, under tonic conditions the compound may elicit a change in the speed of the oscillator (at least in terms of the adaptation to changing LD cycles).

What is claimed is:

1. A method of altering circadian rhythm in a mammal, comprising:
administering to said mammal a PDE5 inhibitor.

2. The method of claim 1, wherein said method further comprises:
testing said mammal for a change in circadian rhythm

3. The method of claim 1, wherein said PDE5 inhibitor is selected from the group consisting of sildenafil, vardenafil, tadalafil and zaprinast.

4. The method of claim 1, wherein said method comprise co-administering said PDE5 inhibitor with a second compound that alters circadian rhythm.

5. The method of claim 1, wherein said method further comprises administering light to said mammal.

6. The method of claim 1, wherein said mammal has, or is expected to have, a circadian rhythm disorder.

7. The method of claim 1, wherein said circadian rhythm disorder is selected form the group consisting of a transmeridian flight disorder (jet-lag), shiftwork-related disorder, seasonal affected disorder and insomnia by phase delay or phase advance.

8. The method of claim 1, wherein said PDE5 inhibitor is administered at a dose of in the range of 25 mg to 250 mg.

9. The method of claim 1, wherein said PDE5 inhibitor is administered as a single dose in prior to a phase change.

10. A method of preventing or treating circadian rhythm disorder in a human, comprising:
administering to said human a PDE5 inhibitor.

11. The method of claim 10, wherein said circadian rhythm disorder is selected form the group consisting of a transmeridian flight disorder (jet-lag), shiftwork-related disorder, seasonal affected disorder and insomnia by phase delay or phase advance.

12. The method of claim 10, wherein said PDE5 inhibitor is administered prior to travel to prevent jet-lag.

13. The method of claim 10, wherein said PDE5 inhibitor is administered late in the evening.

14. The method of claim 10, wherein said PDE5 inhibitor is selected from the group consisting of sildenafil, vardenafil, tadalafil and zaprinast.

15. The method of claim 10, wherein said method comprise co-administering said PDE5 inhibitor with a second compound that alters circadian rhythm.

16. The method of claim 10, wherein said method further comprises administering light to said human.

17. The method of claim 10, wherein said PDE5 inhibitor is administered at a dose of in the range of 25 mg to 250 mg

18. The method of claim 10, wherein said PDE5 inhibitor is administered as a single dose in prior to a phase change.


SEQUENCE LISTING (6 sequences)

SEQ ID NO: 1 - DNA, 20 bp, Artificial Sequence
  Description of Artificial Sequence primer oligonucleotide
caggaaatggtgggaccttc

SEQ ID NO: 2 - DNA, 20 bp, Artificial Sequence
  Description of Artificial Sequence primer oligonucleotide
aaggcttccaggaactgctc

SEQ ID NO: 3 - DNA, 20 bp, Artificial Sequence
  Description of Artificial Sequence primer oligonucleotide
agaacttatccagggcgtgc

SEQ ID NO: 4 - DNA, 20 bp, Artificial Sequence
  Description of Artificial Sequence primer oligonucleotide
tgtcacacagaagcagtgcc

SEQ ID NO: 5 - DNA, 20 bp, Artificial Sequence
  Description of Artificial Sequence primer oligonucleotide
ctgcaccaccacctgcttag

SEQ ID NO: 6 - DNA, 20 bp, Artificial Sequence
  Description of Artificial Sequence primer oligonucleotide
ctttctccaggcgacatgtg